(19) United States
(12) Patent Application Publication
(10) Pub. No.: US 2016/0106539 A1
(43) Pub. Date: Apr. 21, 2016

(54) CARDIAC VALVE SUPPORT DEVICE FITTED WITH VALVE LEAFLETS

(71) Applicant: MVALVE TECHNOLOGIES LTD., Herzliya (IL)

(72) Inventors: Maurice BUCHBINDER, La Jolla, CA (US); Shay DUBI, Tel Aviv (IL); Amit TUBISHEVITZ, Tel Aviv (IL); Avner GEVA, Ra'anana (IL)

(73) Assignee: MVALVE TECHNOLOGIES LTD., Herzliya (IL)

(21) Appl. No.: 14/893,267

(22) PCT Filed: May 27, 2014
(86) PCT No.: PCT/IL14/50476

§ 371 Date: Nov. 23, 2015

Related U.S. Application Data
(60) Provisional application No. 61/828,203, filed on May 29, 2013.
(60) Provisional application No. 61/835,588, filed on Jun. 16, 2013.

(51) Int. Cl. A61F 2/24

(52) U.S. Cl. CPC A61F 2/2412; A61F 2/2409

(57) ABSTRACT

The present invention provides a cardiac valve support device adapted for endovascular delivery to a cardiac valve, wherein said support device comprises either a single support element with valve leaflets connected thereto or two interconnected support elements having valve leaflets connected to one of said two elements. The invention further encompasses a two-stage method for implanting a replacement cardiac valve, wherein the first stage comprises delivering a valve support device fitted with one or more valve leaflets to a location near a subject's cardiac valve, such that said valve leaflets fulfill the function previously fulfilled by the native valve.

### FIELD OF THE INVENTION

The present invention is directed to valve support devices for use in two-stage cardiac valve replacement procedures. More specifically, the present invention provides valve support devices that are fitted with valve leaflets that may be used to maintain valve function prior to the deployment of a permanent replacement valve.

### BACKGROUND OF THE INVENTION

Devices and methods for the two-stage implantation of replacement cardiac valves have been described in co-owned, co-pending international patent application no. PCT/US2011/050232 (published as WO 2012/031141). In the approach disclosed in this publication, a valve support is implanted within the region of a native valve annulus in a first stage, following which, in a second stage, a replacement cardiac valve is implanted within the internal space of said valve support. A key feature of the valve support disclosed in this publication is the presence and orientation of two bridging elements that mutually connect upper and lower ring support elements. These bridging elements are arranged such that the native valve leaflets are able to continue to function subsequent to implantation of the valve support up until implantation of the replacement valve during the second stage of the procedure.

Another type of cardiac valve support device, suitable for use in two-stage implantation methods, is disclosed in co-owned PCT application no. PCT/IL2013/000025, which published as WO 2013/128436. The device described in this publication differs from that of WO 2013/031141 in that it comprises a single annular support element, together with additional stabilizing and sealing elements. However, in common with WO 2013/031141, the valve support device disclosed in this publication is constructed and implanted such that it does not hinder the normal function of the native valve leaflets.

Occasionally, the functionality of the native valve leaflets may become compromised during implantation of the valve support—for example, if the leaflets suffer mechanical damage when the support is manipulated into its working position within the anatomical valve annulus. In such cases, there will be loss of valve function during the period of time that elapses between the cessation of native valve function and the implantation of the replacement valve. This period of time is commonly ten minutes or longer, and the loss of function of, for example, the mitral valve for even much shorter periods of time would be expected to have very serious clinical consequences, such as potentially fatal acute pulmonary edema.

### SUMMARY OF THE INVENTION

The present invention provides an alternative solution to the challenge of maintaining valve function during two step support/valve implantation procedures. In addition, the valve support device provided by the present invention may also be used to maintain valve function for longer periods of time (e.g. in the order of several weeks), thereby enabling the implantation of the replacement valve to be delayed by such periods of time, following the initial deployment of the support device fitted with the valve leaflets. In other cases, the valve leaflets attached to the support device may be used to maintain valve function for even longer periods of time (e.g. over the course of several years), thereby itself acting as a replacement valve.

Thus, in one aspect, the present invention provides a cardiac valve support device that is fitted with one or more valve leaflets that are capable of functioning as a temporary cardiac valve. While most embodiments of the device will be fitted with either two or three leaflets, devices having either larger numbers of such leaflets or a single leaflet only are also within the scope of the present invention.

In one preferred embodiment, the present invention provides a cardiac valve support device adapted for endovascular delivery to a cardiac valve comprising:
first and second support elements each having a collapsed delivery configuration and a deployed configuration;
and wherein at least two bridging members extend from the first support element to the second support element, said bridging members having a delivery configuration and a deployed configuration, wherein said bridging members extend radially inward from the first and second support elements in the deployed configuration;
and wherein one of said support elements is fitted with one or more valve leaflets.

Examples of the basic structure of such an embodiment may be found in co-pending, co-owned international patent application no. PCT/US2011/050232 (published as WO 2012/031141, the entire contents of which are incorporated herein by reference). In such an embodiment the one or more valve leaflets are attached to the upper ring of the valve support device.

In another preferred embodiment, the present invention provides a cardiac valve support adapted for endovascular delivery to a cardiac valve, comprising a single ring-shaped support element, wherein said support element has a collapsed delivery configuration and a deployed configuration, and wherein said support element is fitted with one or more valve leaflets.

Examples of suitable single support element valve support devices, to which valve leaflets may be added, are described in co-pending, co-owned international patent application no. PCT/IL2013/000025 (published as WO 2013/128436, the entire contents of which are incorporated herein by reference).

In both of the above-disclosed preferred embodiments, each of the support elements, in its expanded configuration, generally has the form of a closed ring, the outline shape of which is preferably circular or near-circular. However, these ring elements may also be constructed in any other desired and suitable shape, such as oval, elliptical and so on.

Preferably, the support elements have the form of a flat annular ring.

In a particularly preferred embodiment of the present invention, the valve support device is of a size and shape that permits it to be implanted within the annulus of a mitral valve.

In some preferred embodiments of the present invention, the support elements may be fitted with one or more stabilizing arms, in order to assist with the stabilization of the valve support device within the annulus. These stabilizing arms may be manufactured in a variety of different sizes and shapes. Examples of stabilizing and attachment means (including short arms, elongated arms and wings, and lever mechanisms) that are suitable for use with the support device of the present invention are disclosed in co-pending international applications PCT/US2011/050232 (which published as WO 2012/031141), PCT/IL2013/000025 (which published as WO 2013/128436) and PCT/IL2013/000036 (which published as WO 2013/150512), the contents of which are incorporated herein by reference.

In one preferred embodiment of this aspect of the invention the support device disclosed hereinabove further comprises one or more intra-ventricular stabilizing elements and/or one or more intra-atrial stabilizing elements.

In another aspect, the present invention also provides a two-stage method for implanting a replacement cardiac valve, wherein the first stage comprises delivering a valve support fitted with valve leaflets to a location near a subject's cardiac valve; expanding the support element(s) from a collapsed delivery configuration to an expanded, deployed configuration secured against cardiac tissue in the region of the valve annulus, thereby permitting said valve leaflets to fulfill the function previously fulfilled by the native valve; and wherein the second stage comprises deploying a replacement valve within the central space of said valve support and securing said replacement valve to said valve support. Securing the replacement cardiac valve to the valve support can comprise expanding the replacement cardiac valve from a collapsed delivery configuration to an expanded configuration. Expanding the replacement cardiac valve can include expanding the replacement cardiac valve with a balloon and/or allowing the replacement cardiac valve to self-expand. Securing a replacement cardiac valve to the valve support can comprise securing the replacement cardiac valve radially within the valve support.

The phrase “a location near a subject's cardiac valve” refers to the region of the cardiac valve annulus, and the adjacent regions within the heart.

In a highly preferred embodiment of the method of the invention, the cardiac valve to be replaced is a mitral valve.

In one preferred embodiment of the method, the valve support device comprises as single support element.

In another preferred embodiment of the method, the valve support device comprises an upper support element and a lower support element mutually connected by two or more bridging elements. In this embodiment, the above-disclosed step of expanding the support elements comprises expanding, in sequence, one of the support elements, the bridging elements and the second support element.

In one embodiment, the above-defined method may be employed to deliver the valve support by an endovascular route. In another embodiment, the method may be used to deliver the valve support by a transapical route. Examples of delivery devices suitable for the endovascular and transapical delivery of the above-disclosed valve support are disclosed in co-owned, co-pending international patent application no. PCT/IL2014/050183.

### BRIEF DESCRIPTION OF THE DRAWINGS

FIG. 1 presents a perspective view of a typical two-ring valve support device fitted with valve leaflets.

FIG. 2 depicts the same device as shown in FIG. 1, but with the valve leaflets in their open position.

FIG. 3 shows, in side view, a typical single-ring support device of the present invention fitted with three valve leaflets.

FIG. 4 depicts the same device as shown in FIG. 3, but with the valve leaflets in their open position.

FIG. 5 illustrates a single-ring support device fitted with valve leaflets following deployment and expansion of a replacement cardiac valve within the central space of said support device.

FIG. 6 depicts a two-ring support device fitted with valve leaflets following expansion of a replacement valve.

FIG. 7 shows, in plan view, a single-ring support device of the present invention, having three valve leaflets, shown here in their fully-closed position.

### DETAILED DESCRIPTION OF THE PREFERRED EMBODIMENTS

As explained hereinabove, the valve support device developed by the present inventions provides a solution to the challenge of maintaining valve function during two step support/valve implantation procedures, as well as for longer periods of time (e.g. in the order of several weeks), in cases in which the second stage of the procedure (i.e. implantation of the replacement valve) needs to be delayed by such periods of time.

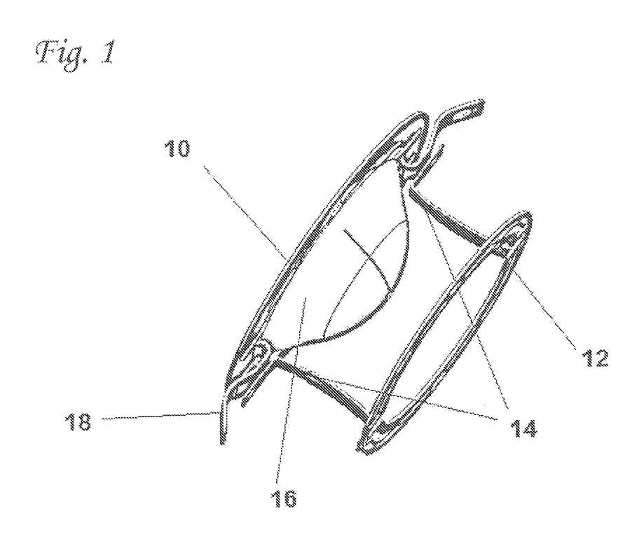

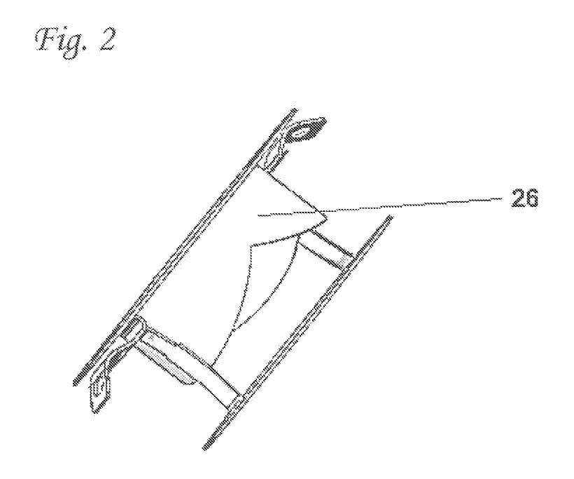

In the two-ring versions of the valve support device (as disclosed hereinabove), the one or more valve leaflets are attached to the upper ring of the valve support device. FIG. 1 shows, in perspective view, an example of such a device, having an upper support element 10 and a lower support element 12 mutually connected by two bridges 14. The valve leaflets 16 in this figure are shown their closed position. Two stabilizing arms 18 attached to upper support element 10 are also shown. The same embodiment, but with the valve leaflets 26 in their open position, is illustrated in FIG. 2.

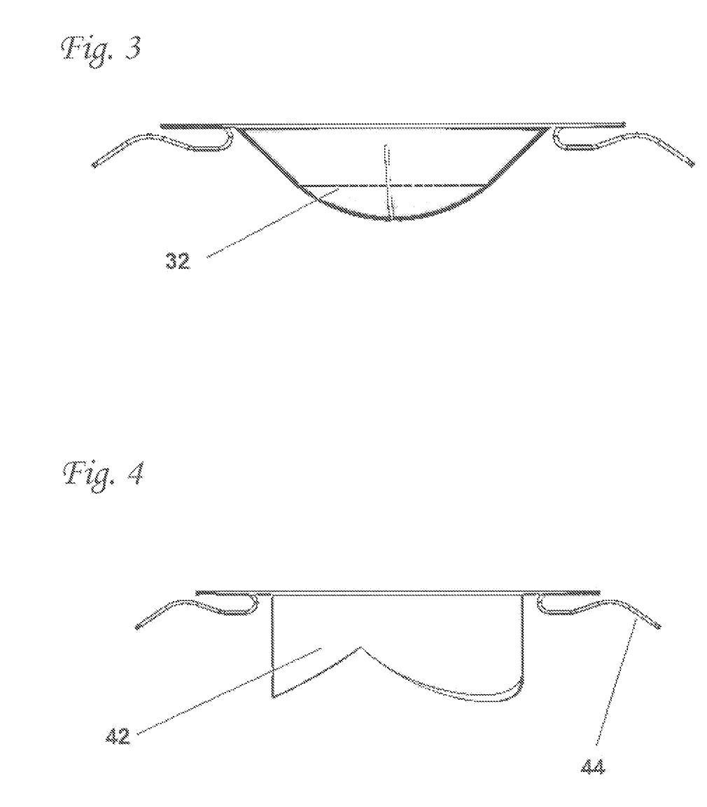

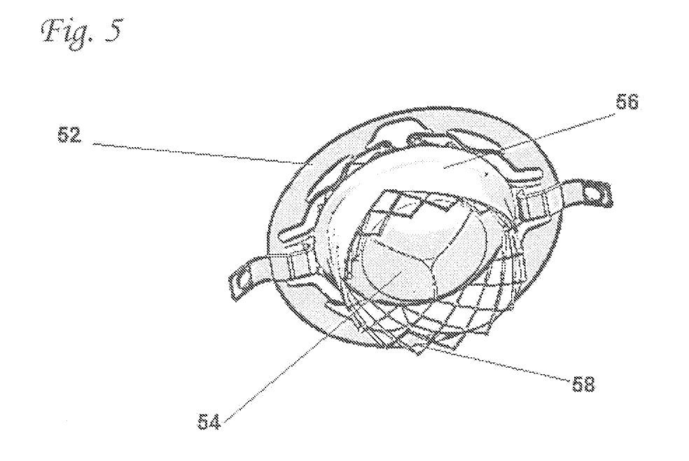

In the single-ring version of the valve support device of the present invention, the one or more valve leaflets are attached to the single support ring. FIG. 3 depicts, in perspective view, an example of such a single-support device in which the three valve leaflets 32 attached thereto are in their fully-closed position. The same embodiment, but with the valve leaflets 42 fully open, is shown in FIG. 4.

A key feature of the valve support device of the present invention is the fact that it is constructed such that it may be adopt two different, stable configurations: a collapsed configuration that permits the delivery of the device via a catheter that is passed through the patient's vasculature; and a second expanded configuration that the device adopts when it is caused to leave the confines of the delivery catheter during implantation within the cardiac valve annulus.

In the case of the two-ring support element embodiments the height of the valve support, measured from the base of the first support to the top of the second support, is generally in the range of about 1 cm to about 5 cm to be able to accommodate the height of the replacement heart valve, such as a stented heart valve. In some embodiments the height is greater than 5 cm. In some embodiments the height of the valve support is between about 1 cm and about 2.5 cm. For example, a stented heart valve in an expanded configuration can have a height of about 17.5 mm. It should be noted, of course, that these numbers are merely exemplary and are not limiting in any way.

In some embodiments the first and second support elements and the bridge members are made from a resilient material that can be deformed into a delivery configuration yet are adapted to self-expand to an expanded configuration, with optional additional expansion of one or more components by balloon dilation. For example, the support can be made from Nitinol, relying on its superelastic properties. In some embodiments the valve support is made from a material with shape memory properties, such as Nitinol, and is adapted to return to an expanded memory configuration after being heated above its transition temperature. In some embodiments in which the valve support is made from a material such as Nitinol, the shape memory properties and the superelastic properties are utilized.

In one preferred embodiment of the valve support device of the present invention the outer perimeter of at least one of the two support elements (or in the case of the single-ring support element, the only such element) is entirely rigid, such that when released from the delivery catheter, it is not possible to cause further radial expansion of the outer diameter of said device.

In the radial plane (i.e. the plane in which the native cardiac valve leaflets are disposed when in their closed position), the size of at least one of the support elements (the upper of the two elements, after deployment, or the only support element in the case of the single-ring device) may be defined in terms of its outer radius (Ro), its inner radius (Ri) and the difference between these two radii (Rd). It should be appreciated that Ro is determined by the diameter of the mitral valve annulus into which the valve support device will be implanted. Ri, however, is determined by the outer diameter of the replacement heart valve that will be inserted into the central space of the support device. Generally, the prosthetic aortic valves used in conjunction with the valve support device of the present invention have an external diameter considerably less than that of the mitral valve annulus. Thus, typically, the outer diameter of the support element will be in the range of 30-50 mm, while the inner diameter will have a value in the range of 23-29 mm. It may therefore be appreciated that Rd approximately corresponds to the annular gap between the small outside-diameter replacement valve and the relatively large diameter mitral valve annulus. Thus, in one preferred embodiment, at least one of the support elements in its deployed configuration is provided in the form of a flat annular ring, wherein the difference between the outer radius and the inner radius of said annular ring (Rd) is in the range of 1-20 mm. With regard to the thickness of the support element (t) (as measured along the longitudinal axis of the element when in situ), t represents a compromise between the need for minimizing this parameter in order to facilitate crimping and insertion into a delivery catheter, and the need for the support device to be sufficiently rigid such that it is able to withstand the forces exerted by the beating heart without buckling. The thickness of the at least one of the two support elements (or of the single support element in the single-ring device) is generally in the range of 0.25-0.8 mm, more preferably 0.4 mm. In one typical, non-limiting example, t is 0.4 mm, while Rd has a value of 5.5 mm. Indeed, as a general rule, in most embodiments of the annular support element of the present invention, Rd is significantly larger than t. For example, in many cases Rd may be between 2.5 and 35 times larger than t, more preferably between 10 and 20 times larger than t. It may be appreciated from the foregoing explanation that the ratio between Rd and t has functional significance for the valve support device of the present invention.

The valve leaflets that are fitted to the support device are preferably constructed from biocompatible non-biological materials such as polyurethane, Nylon, Dacron, Teflon, Nitinol, and so on. It is well known in the art that leaflets made of such materials are generally unsuitable for long-term use in prosthetic valves. However, for the purpose of the present invention—that is, replacement of native cardiac valve function for periods of time in the range of a few minutes to a few weeks—said materials provide sufficient suitability and biocompatibility. Furthermore, leaflets constructed from non-biological materials (such as those mentioned hereinabove, have the following additional advantages when compared with leaflets prepared from biological materials:
Lower manufacturing cost
Simpler production process
No danger of infection being transmitted from foreign biological tissue to the patient
Less complex regulatory procedures.

In other embodiments, the valve leaflets may be constructed from biological materials such as pericardium (e.g. bovine, equine or porcine) or from biological valves from mammalian subjects. Leaflets prepared from such biological sources may be advantageous in certain circumstances.

The leaflets (non-biological or biological) may be attached to the support device by any suitable method known to the skilled artisan including, but not limited to, suturing, welding (for example laser welding, ultrasonic welding etc.), adhesion with biocompatible glues.

As indicated hereinabove, in some preferred embodiments of the present invention, the support elements may be fitted with one or more stabilizing arms, in order to assist with the stabilization of the valve support device within the annulus. These stabilizing arms may be manufactured in a variety of different sizes and shapes. In the example depicted in FIG. 4 the stabiliizing arms 44 (shown attached to the inferior surface of a single-support device) are relativlely short and are formed with multiple curvatures.

The valve support device of the present device may also further comprise additional structural features aimed at improving the stability and increasing the efficiency of the permanent replacement valve, including: an elastically deformable inner perimeter, guidance elements for use in centering a guide wire that is passed through the center of said support device, pressure release means comprising at least one reduced diameter segment in the outer circumference of at least one of the support elements and paravalvular sealing drapes. Details of all of these features are fully-disclosed in co-owned, co-pending international patent applications PCT/IL2013/000025 and PCT/IL2013/000036, the contents of which are incorporated herein by reference.

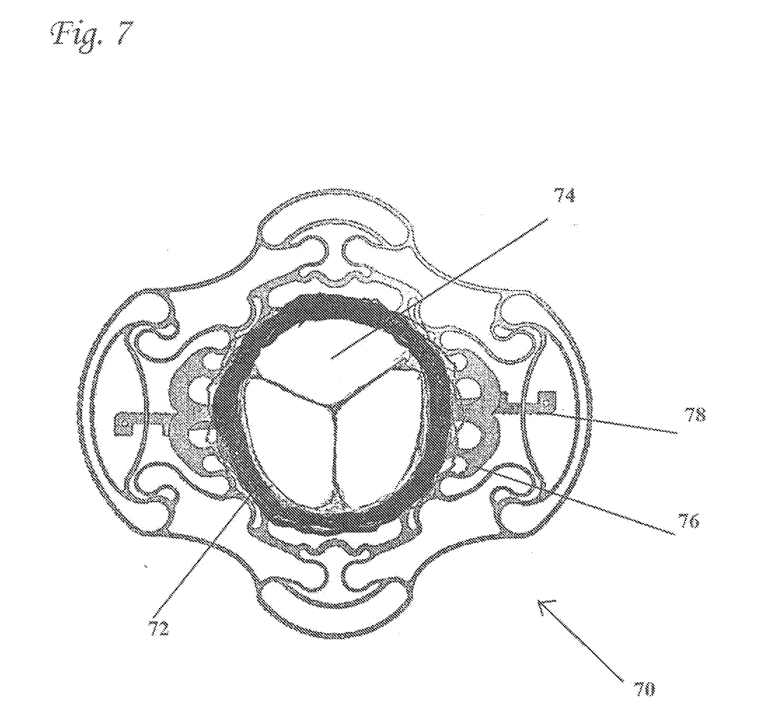

In addition, in some embodiments the valve support of the present invention may further comprise one or more lateral extensions as a means for reducing paravalvular leakage as well as improving the co-axial positioning of the device. These extensions have a surface area which essentially extends the surface area of the ring laterally outwards, to the outer aspect of the ring. The length and width of the extension in the plane of the ring (the lateral plane) are significantly larger than the thickness of the extension, that is, the dimension measured along the longitudinal plane (which is typically only the width of the wire or sheet from which the extension was made). In some embodiments, the extension elements are not continuously connected to the external aspect of the ring along their entire length, but rather are connected to the ring only at discrete singular connection points (for example, connected only at two points, on at the front edge of said element and one at the back edge thereof), without any connection at the central part of the element. In other embodiments, the lateral extensions from a complete crown-like structure around the outer edge of the valve support device. The lateral extensions are deployed on the atrial side of the mitral annulus, above the commissures of the mitral valve, in such a way that they “cover” the space formed by the commissures. FIG. 7 provides an example of a single-ring support device of the present invention 70 which comprises, in addition to three valve leaflets 74 attached to support ring 72, a crown-like lateral extension structure encompassing the entire outer aspect of said support ring. The lateral free ends of two anchoring wings 78 are also seen in this figure.

As disclosed hereinabove, the present invention also provides a two-stage method for implanting a replacement cardiac valve, wherein the first stage comprises delivering a valve support fitted with valve leaflets to a location near a subject's cardiac valve; expanding the support element(s) from a collapsed configuration to an expanded, deployed configuration secured against cardiac tissue in the region of the valve annulus, thereby permitting said valve leaflets to fulfill the function previously fulfilled by the native valve; and wherein the second stage comprises securing a replacement valve to the valve support.

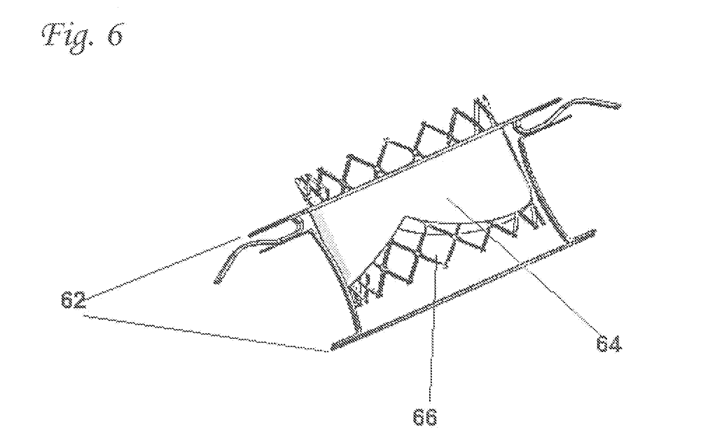

During deployment of the replacement cardiac valve, the expansion of said valve causes the lateral displacement of the valve leaflets, thereby disabling them, the cardiac valve function now be solely fulfilled by the leaflets of the deployed replacement cardiac valve. An example of a support device having a single support ring 52 following deployment and expansion of the replacement valve is shown in FIG. 5. In this figure, the permanent replacement valve leaflets 54 are shown in their fully-closed position, whilst the valve leaflets 56 attached to the support device have been displaced laterally by the expanded stent portion 58 of the replacement valve, such that said valve leaflets 56 are now disabled. Similarly, FIG. 6 presents a side view of a support device having two interconnected support elements (rings) 62 with the support device valve leaflets 64 disabled, following expansion of the stent portion 66 of a replacement valve (permanent valve leaflets not shown for clarity).

In a particularly preferred embodiment of the method of the present invention, the replacement cardiac valve is a prosthetic aortic valve. Examples of suitable commercially-available prosthetic aortic valves include (but are not limited to): Sapien Valve (Edwards Lifesciences Inc., US), Lotus Valve (Boston Scientific Inc., US), CoreValve (Medtronic Inc.) and DFM valve (Direct Flow Medical Inc., US).

In addition to replacing the functionality of the native leaflets during the two-step replacement procedure described hereinabove, the valve support device fitted with valve leaflets of the present invention has several other advantages. One such advantage relates to the fact that in contradistinction to certain prior art devices (such as those disclosed in co-owned, co-pending international patent applications PCT/US2011/050232 (which published as WO 2012/031141), there is no need to implant the valve support device in such a way that the native valve leaflets may continue to function. Thus, while in said prior art devices the stabilization and attachment means (such as arms, wings and lever mechanisms) need to be aligned such that they are located in the region of the native valve commissures only (in order not to interfere with the opening and closure of the native leaflets), the valve support of the present invention may also incorporate stabilizing and attachment means that engage or make contact with the native leaflets.

Additionally, the lack of dependence on native leaflet function also provides the opportunity to orientate the valve support device in the first stage of the claimed method (and hence the orientation of the permanent replacement valve in the second stage thereof) at any desired angular rotation. Thus, in one embodiment of the method of the present invention, a valve support device fitted with two leaflets is implanted within the native annulus in such a way that the commissural line of said support device leaflets is orientated at 90 degrees to the native valve commissure.

Further advantages associated with the use of the valve support of the present invention are seen during the second phase of the valve replacement procedure, i.e. during the implantation of the prosthetic valve within the central space of the annular valve support. Thus, the presence of the support device leaflets—which are displaced outwards by the implanted prosthetic valve—increases the frictional resistance offered by the support device, and therefore assists in the retention of said prosthetic valve within said support. Similarly, the outwardly-displaced valve leaflets also assist in reducing paravalvular leakage.

1. A cardiac valve support adapted for endovascular delivery to a cardiac valve, comprising:
first and second support elements each having a collapsed delivery configuration and a deployed configuration;
and wherein at least two bridging members extend from the first support element to the second support element, said bridging members having a delivery configuration and a deployed configuration, wherein said bridging members extend radially inward from the first and second support elements in the deployed configuration;
and wherein one of said support elements is fitted with one or more valve leaflets.

2. A cardiac valve support adapted for endovascular delivery to a cardiac valve, comprising a single ring-shaped support element, wherein said support element has a collapsed delivery configuration and a deployed configuration, and wherein said support element is fitted with one or more valve leaflets.

3. The cardiac valve support according to claim 1, wherein said support further comprises one or more stabilizing arms.

4. The cardiac valve support according to claim 1, wherein said support is fitted with three valve leaflets.

5. The cardiac valve support according to claim 1, wherein said support is fitted with two valve leaflets.

6. The cardiac valve support according to claim 1, wherein the one or more valve leaflets are constructed from a non-biological material.

7. The cardiac valve support according to claim 1, wherein at least one of the support elements in its deployed configuration has the form of a flat annular ring, and wherein the difference (Rd) between the outer radius and the inner radius of said annular ring is in the range of 1-20 mm.

8. The cardiac valve support according to claim 7, wherein the ratio between Rd and the thickness of the flat annular ring is between 10:1 and 20:1.

9. The cardiac valve support according to claim 7, wherein the inner diameter of the flat annular ring is in the range of 23-29 mm and the outer diameter thereof is in the range of 30-50 mm.

10. The cardiac valve support according to claim 7, wherein the thickness of the flat annular ring is in the range of 0.25-0.8 mm.

11. A two-stage method for implanting a replacement cardiac valve,
wherein the first stage comprises delivering a valve support device comprising one or more support elements and one or more valve leaflets attached to one of said support elements, to a location near a subject's cardiac valve, expanding said support element(s) from a collapsed delivery configuration to an expanded, deployed configuration secured against cardiac tissue in the region of the valve annulus, thereby permitting said valve leaflets to fulfill the function previously fulfilled by the native valve;
and wherein the second stage comprises deploying a replacement valve within the central space of said valve support and securing said valve thereto.

12. The method according to claim 11, wherein the valve support device comprises a single support element.

13. The method according to claim 11, wherein the valve support device comprises an upper support element and a lower support element mutually connected by two or more bridging elements, and wherein the step of expanding said support elements comprises expanding, in sequence, one of the support elements, the bridging elements and the second support element.

14. The method according to claim 11, wherein the cardiac valve to be replaced is the mitral valve.
